(19) United States
(12) Patent Application Publication
(10) Pub. No.: US 2022/0000817 A1
(43) Pub. Date: Jan. 6, 2022

(54) PROGNOSIS AND TREATMENT OF METASTATIC CANCER

(71) Applicant: Academia Sinica, Taipei (TW)

(72) Inventors: Wei-Chao CHANG, Taichung City (TW); Chung-Hsuan CHEN, Taipei (TW)

(73) Assignee: Academia Sinica, Taipei (TW)

(21) Appl. No.: 17/289,736

(22) PCT Filed: Nov. 1, 2019
(86) PCT No.: PCT/US2019/059561

§ 371 Date: Apr. 29, 2021

Related U.S. Application Data
(60) Provisional application No. 62/754,549, filed on Nov. 1, 2018.

(51) Int. Cl. A61K 31/192; C12N 15/113; A61K 31/7105; C12N 15/115; C07K 16/18; A61P 35/04

(52) U.S. Cl. CPC A61K 31/192; C12N 15/113; A61K 31/7105; C12N 2310/14; C07K 16/18; A61P 35/04; C12N 2310/531; C12N 15/115

(57) ABSTRACT

Provided herein is a method for treating a subject afflicted with a metastatic cancer. The method includes, administering to the subject an effective amount of an agent capable of suppressing the expression of a nucleic of EF-hand domain-containing protein D2 (EFHD2) or a polypeptide encoded by the nucleic acid in the metastatic cancer of the subject. The agent may be a 2-aryl propionic acid (2-APA) compound, or a short hairpin ribonucleic acid (shRNA) that directs cleavage of EFHD2 gene RNA via RNA interference. Also provided herein is a method for detecting and/or diagnosing whether a subject having a metastatic cancer via a biological sample of the subject. The method includes steps of, measuring the level of EFHD2 nucleic acid or polypeptide in the biological sample; and comparing the amount of the EFHD2 nucleic acid or polypeptide present in the biological sample with that of a healthy subject; in which an elevated amount of EFHD2 nucleic acid or polypeptide in the biological sample relative to that of a healthy subject indicates that the cancer of the subject is likely to metastasize.

### CROSS-REFERENCE TO RELATED APPLICATIONS

This application is a national stage filing under 35 U.S.C. 371 of International Application PCT/US2019/059561, filed Nov. 1, 2019, which claims priority to U.S. Ser. No. 62/754,549 filed Nov. 1, 2018; the disclosure of afore-indicated prior applications are incorporated herein by reference in their entireties.

### BACKGROUND OF THE INVENTION

### 1. Field of the Invention

The present disclosure in general relates to prognosis and/or treatment of a cancer, particularly, a metastatic cancer.

### 2. Description of Related Art

Metastasis is complex series of steps in which neoplastic cells leave the original tumor site and migrate to other parts of the body via blood stream or the lymphatic system and starts new tumors that resemble the primary tumor. It would be life-saving to predict or diagnose in advance whether a primary cancer has the potential to metastasize, so that high risk patients can be subject to close follow up or specific treatment regime that varies with the metastasized cancer.

Accordingly, there exists in the related art, a need of a method for rendering a prognosis on whether the cancer of a subject is likely to metastasize, and a need of a method for treating a subject having a metastatic cancer.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In general, the present disclosure relates to the unexpected discovery that the expression of EF-hand domain-containing protein D2 (EFHD2) is positively correlated to metastasis of tumors.

Accordingly, the present disclosure provides novel small hairpin ribonucleic acids (shRNAs) useful for preventing and/or treating metastatic tumor; as well as novel methods of detecting, suppressing, predicting, and/or treating tumor metastasis based on the presence or absence of EFHD2 in the tumor.

The first aspect of the present disclosure aims at providing an isolated double stranded short hairpin ribonucleic acid (shRNA) that directs cleavage of EFHD2 gene RNA via RNA interference, wherein one strand of said shRNA molecule comprises a ribonucleic acid complementary to said EFHD2 gene RNA encoded by a nucleic acid of SEQ ID NO:1 or a portion thereof.

According to embodiments of the present disclosure, the shRNA has the ribonucleic acid at least 90% identical to SEQ ID NO: 4.

The second aspect of the present disclosure aims at providing a method for treating a subject afflicted with a metastatic cancer. The method comprises the step of, administering to the subject an effective amount of an agent capable of suppressing the expression of a nucleic of EFHD2; or a polypeptide encoded by the nucleic acid, in the metastatic cancer of the subject.

According to some embodiments of the present disclosure, the agent is an isolated double stranded shRNA that directs the cleavage of the ribonucleic acid of EFHD2. Preferably, the shRNA has the ribonucleic acid sequence at least 90% identical to SEQ ID No: 4, which is encoded by a nucleic acid of SEQ ID NO: 1 or a portion thereof.

According to other embodiments of the present disclosure, the agent is an inhibitor specific to EFHD2. In some embodiments, the inhibitor is an antibody capable of binding selectively to EFHD2 polypeptide. In further embodiments, the inhibitor is an aptamer identified through SELEX (Systematic Evolution of Ligands by Exponential Enrichment) and is capable of binding specifically to EFHD2 polypeptide. In other embodiments, the inhibitor is a 2-aryl propionic acid (2-APA) selected from the group consisting of ibuprofen, naproxen, flurbiprofen, and ketoprofen.

According to some embodiments of the present disclosure, the method further comprises the step of, administering to the subject another agent capable of evoking the expression of caveolin-1 (CAV1).

Examples of the metastatic cancer includes, but is not limited to, breast cancer, gastric cancer, gastrointestinal stromal tumor (GIST), lung cancer (e.g., non-small cell lung cancer (NSCLC)), and pancreatic cancer.

The present disclosure also aims at providing a method of making a prognosis on whether a subject has a metastatic cancer via a biological sample of the subject. The method includes steps of:

measuring the level of EFHD2 nucleic acid or polypeptide in the biological sample; and

comparing the amount of the EFHD2 nucleic acid or polypeptide present in the biological sample with that of a healthy subject;

wherein, an elevated amount of EFHD2 nucleic acid or polypeptide in the biological sample relative to that of a healthy subject indicates that the cancer of the subject is likely to metastasize.

According to embodiments of the present disclosure, the biological sample may be any of a tissue biopsy sample, a whole blood sample, a plasma sample, a serum sample, a urine sample, or a mucus sample.

According to embodiments of the present disclosure, the cancer may be any of breast cancer, gastric cancer, gastrointestinal stromal tumor (GIST), lung cancer (e.g., non-small cell lung cancer (NSCLC)), or pancreatic cancer.

According to embodiments of the present disclosure, the subject has gone through prior cancer removal surgery.

Many of the attendant features and advantages of the present disclosure will become better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIG. 1. EFHD2 increases the metastatic abilities of lung adenocarcinoma cells. The effects of EFHD2 overexpression in A549 cells and EFHD2-knockdown in H1299 cells on metastatic abilities were determined. (A) Migration ability was analyzed by wound-healing assay. (B) Invasive ability was analyzed by transwell invasion assay. (C) Invadopodia were visualized by colocalized of cortactin (green) and F-actin (red). (D) Quantification of cancer cells with invadopodia. N=100 cells/sample. **, P<0.01.

FIG. 2 EFHD2 promotes the EMT. Western blot assay was used to determine the protein expression of EMT-related markers E-cadherin and vimentin in (A) EFHD2-knockeddown H1299 and H2981 cells and (B) EFHD2-overexpressing A549 and CL1-0 cells. β-actin, loading control.

FIG. 3 EFHD2 promotes the EMT partly through inhibition of CAV1. (A) The effect of EFHD2 on CAV1 expression in A549 and CL1-0 cells was determined by Western blot assays. β-actin, loading control. (B) CAV1 protein in EFHD2-overexpressing A549 cells and its control was analyzed by confocal microscopy. (C) CAV1 mRNA levels in EFHD2-overexpressing A549 cells and its control were analyzed by qPCR. (D) The effect of CAV1 knockdown in A549 cells and CAV1 re-expression in EFHD2-overexpressing A549 cells on E-cadherin and vimentin levels was determined by Western blot assay.

FIG. 4 2-APAs, but not NSAIDs, suppress EFHD2 expression in cancer cells. The effect of (A) 2-APA compounds, including ibuprofen, naproxen, flurbiprofen, and ketoprofen; and (B) NSAID compounds, including aspirin, diclofenac, ketorolac, mefenamic acid, piroxicam, and sulindac, on EFHD2 in H1299 and F4 cells were determined by Western blot assay. β-actin, loading control.

FIG. 5 Ibuprofen reduced EFHD2 expression in cancer cells. (A) The effect of various doses of ibuprofen on EFHD2 in H1299 and F4 cells were respectively determined by Western blot assay; (B) The effects of ibuprofen on the invasion and migration ability of H1299 and F4 cells were analyzed by matrigel transwell system.

FIG. 6. Ibuprofen activates both proteasomal and lysosomal degradation of EFHD2. H1299 cells were pretreated with MG132 and/or Baf-A1 for 0.5 hr, then followed by the treatment of 600 μM ibuprofen for the indicated time. EFHD2 expression was determined by western blot assay.

FIG. 7 Ibuprofen enhanced susceptability of cancer cells to a chemotherapeutic drug. MTT assay was used to determine cell survival ratio in the control cells (H1299^{shGFP}) or cells having the expression of EFHD2 being knock-out via interference RNA (H1299^{shEFHD2}) with or without the treatment of ibuprofen and/or cisplatin.

FIG. 8 Susceptibility of cancer cells lacking endogenous EFHD2 to chemotherapeutic drug is not enhanced by ibuprofen. MTT assay was used to determine cell survival ratio in the control cells (A549^{pcDNA}) or cells transfecting with vectors that carrying exogenous EFHD2 gene (A549^{pEFHD2}) with or without the treatment of ibuprofen and/or cisplatin.

FIG. 9 Synergistically reduced tumor size via the combined treatment of ibuprofen and cisplatin. (A) are photographs of tumors taken out from xenograft mice treated with cisplatin, ibuprofen and a combination of cisplatin and ibuprofen. (B) is a line graph summarizing the change in tumor volume of xenograft mice in panel (A) along with time.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definitions

For convenience, certain terms employed in the context of the present disclosure are collected here. Unless defined otherwise, all technical and scientific terms used herein have the same meaning as commonly understood by one of the ordinary skill in the art to which this invention belongs.

The term “expression” as used herein is intended to refer to transcription of a gene when a condition is met, resulting in the generation of mRNA and usually encoded protein. Expression can be achieved or performed naturally by the cell (i.e., without artificially intervention) or may be achieved or performed artificially (i.e., with the involvement of artificially intervention, such as by the use of promoters regulated by the use of a chemical agent). The expression may also be initiated by a recombination event triggered by a site-specific recombinase, such as by Cre-mediated recombination. Expression may be determined by measuring mRNA transcribed from the gene or by measuring protein encoded by the gene.

The term “nucleic acid” refers to polynucleotides such as deoxyribonucleic acid (DNA) and where appropriate, ribonucleic acid (RNA). Nucleic acids include but are not limited to single-stranded and double-stranded polynucleotides. Illustrative polynucleotides include DNA, single-stranded DNA, cDNA, and mRNA. The term also includes, analogs of either DNA or RNA made from nucleotide analogs, and as applicable, single (sense or antisense) and double-stranded polynucleotides. The term further includes modified polynucleotides, including modified DNA and modified RNA, e.g., DNA and RNA comprising one or more unnatural nucleotide or nucleoside. The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, and/or which have similar binding properties as the reference nucleic acid, and/or which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also encompasses conservatively modified variants thereof (e.g., degenerate codon substitutions) and complementary sequences, as well as the sequence explicitly indicated.

The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. The polypeptides described herein are not limited to a specific length of the product; thus, peptides, oligopeptides, and proteins are included within the definition of polypeptide, and such terms may be used interchangeably herein unless specifically indicated otherwise. The polypeptides described herein may also comprise post-expression modifications, such as glycosylations, acetylations, phosphorylations and the like, as well as other modifications known in the art, both naturally occurring and non-naturally occurring. A polypeptide may be an entire protein, or a subsequence, fragment, variant, or derivative thereof.

The term “identical” or “percent identity” as used herein refers to two or more sequences or subsequences that are the same or have a specified percentage of nucleotides (or amino acid residues) that are the same, when compared and aligned for maximum correspondence. To determine the percent identity, the sequences are aligned for optimal comparison purposes (e.g., gaps can be introduced in the sequence of a first sequence for optimal alignment with a second sequence). The nucleotides (or amino acid residues) at corresponding nucleic acid (or amino acid) positions are then compared. When a position in the first sequence is occupied by the same nucleotide (or amino acid residue) as the corresponding position in the second sequence, then the molecules are identical at that position. The percent identity between the two sequences is a function of the number of identical positions shared by the sequences (i.e., % identity (number of identical positions/total number of positions)*100). Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. In certain embodiments, the two sequences are of the same length. Thus, 100% identity means, for example, that upon comparing 20 sequential amino acid residues in two molecules respectively having the same or different numbers of residues, both 20 residues in the two different molecules are identical.

The term “treatment” as used herein are intended to mean obtaining a desired pharmacological and/or physiologic effect, e.g., delaying or inhibiting the metastasis of a cancer. The effect may be prophylactic in terms of completely or partially preventing a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., a cancer or heart failure) from occurring in an individual who may be pre-disposed to the disease but has not yet been diagnosed as having it; (2) inhibiting a disease (e.g., by arresting its development); or (3) relieving a disease (e.g., reducing symptoms associated with the disease).

The term “administered”, “administering” or “administration” are used interchangeably herein to refer a mode of delivery, including, without limitation, intraveneously, intramuscularly, intraperitoneally, intraarterially, intracranially, or subcutaneously administering an agent (e.g., an antibody of EFHD2 or a short hairpin ribonucleic acid (shRNA)) that interferes the expression of EFHD2 gene.

The term “an effective amount” as used herein refers to an amount effective, at dosages, and for periods of time necessary, to achieve the desired result with respect to the treatment of a metastatic cancer. For example, in the treatment of a metastatic cancer, an agent (i.e., the antibody of EFHD2 or the small ribonucleic acid that interferes the expression of EFHD2 RNA) which decrease, prevents, delays or suppresses or arrests the expression of EFHD2 would be effective in preventing cancer cells from spreading to other locations and/or from growing. An effective amount of an agent is not required to cure a disease or condition but will provide a treatment for a disease or condition such that the onset of the disease or condition is delayed, hindered or prevented, or the disease or condition symptoms are ameliorated. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the like. Effective amount may be expressed, for example, as the total mass of the active agent (e.g., in grams, milligrams or micrograms) or a ratio of mass of the active agent to body mass, e.g., as milligrams per kilogram (mg/kg). The effective amount may be divided into one, two or more doses in a suitable form to be administered at one, two or more times throughout a designated time period.

The term “subject” or “patient” is used interchangeably herein and is intended to mean a mammal including the human species that is treatable by the compound of the present invention. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, rat, mouse, guinea pig, monkey, pig, goat, cow, horse, dog, cat, bird and fowl. In a preferred embodiment, the subject is a human.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a,” “and,” and “the” are used herein to include plural referents unless the context clearly dictates otherwise.

### 2. Detail Description of Preferred Embodiments

2.1 Methods and/or Kits for Making Prognosis on Cancer Metastasis

The present disclosure is based, at least in part, on the unexpected discovery that the expression of EF-hand domain-containing protein D2 (EFHD2) is positively correlated with tumor metastasis. Accordingly, the present disclosure provides a diagnostic or prognostic biomarker (i.e., EFHD2) capable of distinguishing between metastatic or non-metastatic cancers, as well as detecting and monitoring metastatic cancer cells during therapy. Further, agents that suppress the expression of EFHD2 are potential candidates for the development of medicaments for the treatment and/or prophylaxis of cancer, particularly, metastatic cancers.

According to the present disclosure, there is provided a method of making a prognosis on whether a subject has a metastatic cancer via a biological sample of the subject. The method includes steps of: measuring the level of EFHD2 nucleic acid or polypeptide in the biological sample; and comparing the amount of the EFHD2 nucleic acid or polypeptide present in the biological sample with that of a healthy subject; wherein, an elevated amount of EFHD2 nucleic acid or polypeptide in the biological sample relative to that of a healthy subject indicates that the cancer of the subject is likely to metastasize.

According to embodiments of the present disclosure, the subject is or has been afflicted with cancer, which may be any of breast cancer, gastric cancer, gastrointestinal stromal tumor (GIST), lung cancer (e.g., non-small cell lung cancer (NSCLC)), or pancreatic cancer. According to some embodiments of the present disclosure, the subject has gone through prior surgery to remove the cancer.

To implement the present method, a biological sample is first taken from the subject. Examples of the biological sample suitable for use in the present method include, but are not limited to, a tissue biopsy sample, a whole blood sample, a plasma sample, a serum sample, a urine sample, and a mucus sample. According to preferred embodiments of the present disclosure, a lung tissue biopsy sample is used for rendering a prognosis.

According to embodiments of the present disclosure, the measurement of the level of EFHD2 nucleic acid refers to the measurement of EFHD2 mRNA level, which may be determined by any assays commonly used or known to persons having ordinary skill in the art. To this purpose, total RNA in a biological sample is extracted by use of a chemical solution with high corrosiveness (e.g., phenol, trichloroacetic acid/acetone, and Trizol) followed by neutralization with chloroform. The mixture is centrifuged, and the aqueous phase that contains the extracted RNA is precipitated by an organic solution, such as ethanol and isopropanol. The extracted RNA is then washed with ethanol to remove any contaminated protein, then subjected to drying (e.g., air dry and vacuum dry) to produce RNA pellet. The RNA pellet is then dissolved in diethylpyrocarbonate-treated H₂O (DEPC H₂O), and converted into corresponding cDNA by reverse transcription (RT). In general, RT is performed by mixing the RNA with primer Oligo(dT)₂₀, deoxy-ribonucleoside triphosphate (dNTP, which comprises dATP, dGTP, dTTP, and dCTP), reverse transcriptase, reaction buffer, and optionally, the co-factor of reverse transcriptase (e.g., MgCl₂). Preferably, the reaction mixture further comprises dithiothreitol (DTT), a redox reagent used to stabilize the reverse transcriptase, and RNase inhibitor preventing the degradation of RNA during RT. The cDNA serving as a template may then be quantified by quantitative polymerase chain reaction assay (qPCR) or microarray (e.g., cDNA array and oligonucleotide array). According to one specific example, the mRNA level of EFHD2 is measured by qPCR.

According to other embodiments of the present disclosure, instead of measuring EFHD2 nucleic acid level in the biological sample, EFHD2 polypeptide level is determined. For example, the biological sample may be incubated with an antibody of EFHD2 under conditions that allow for formation of an immunological complex, which is then visualized and/or quantified by western blot analysis, enzyme-linked immunosorbent assay (ELISA), immunohistochemistry (IHC) assay, immunocytochemistry (ICC) assay, immunofluorescence (IF) assay, or luminex assay. According to one working example, the protein level of EFHD2 is determined by IHC.

Based on the measured EFHD2 nucleic acid or polypeptide level in the biological sample, a physician or a clinical practitioner may compare the measured level with that of a healthy subject (i.e., a subject that is free of cancer), and determine whether the biological sample is a cancerous sample, and whether the cancer on the subject is likely to metastasize. According to embodiments of the present disclosure, an elevated level of EFHD2 nucleic acid or polypeptide in the biological sample as compared with that of a healthy sample is an indication that the biological sample is a cancerous sample, and the cancer on the subject is likely to metastasize.

The present disclosure thus contemplates a kit, which is capable of measuring the level of EFHD2 nucleic acid or polypeptide in a biological sample.

According to some embodiments, the components included in the kits may be: a container; an antibody of EFHD2; at least one agent suitable for detecting the binding of EFHD2 with the anti-EFHD2 antibody in the biological sample; and a legend associated with the container and indicating how to use the antibody for detecting EFHD2 in the biological sample. According to other embodiments. The components included in the kits may be: a container; primers suitable for amplifying the nucleic acid of EFHD2; reagents suitable for use in PCR; and a legend associated with the container and indicating how to use the primers for detecting and/or amplifying EFHD2 in the biological sample. The legend may be in a form of pamphlet, CD, VCD, DVD or a software application. The kit may further comprise a negative control that indicates the normal level of EFHD2 nucleic acid or polypeptide in a healthy subject.

2.2 Methods for Treating and/or Preventing Metastatic Cancers

The present disclosure also aims at providing a therapeutic and/or prophylactic treatment to a subject having a metastatic cancer. To this purpose, agents capable of suppressing the expression of EFHD2 are provided and used as medicaments for preventing and/or treating metastatic cancers.

The present disclosure thus encompasses a method for treating a subject afflicted with a metastatic cancer. The method comprises, administering to the subject an effective amount of an agent capable of suppressing the expression of a nucleic of EFHD2; or a polypeptide encoded by the nucleic acid, in the metastatic cancer of the subject.

According to one preferred embodiment, an isolated double stranded shRNA capable of directing the cleavage of EFHD2 gene RNA via RNA interference is used as the agent. The shRNA is characterized in having one strand of the ribonucleic acid complementary to the EFHD2 gene RNA. According to embodiments of the present disclosure, the shRNA is encoded by a nucleic acid of SEQ ID NO: 1 or a portion thereof, and has the ribonucleic acid at least 90% identical to SEQ ID NO: 4.

According to another embodiment of the present disclosure, an inhibitor specific to EFHD2 is used as the agent. The inhibitor may be an antibody or an aptamer identified through SELEX (Systematic Evolution of Ligands by Exponential Enrichment), the antibody or the aptamer is capable of binding selectively to EFHD2 polypeptide and prevents it from exerting any biological activity, such as activating any other cellular proteins that lead to the metastasis of cancer. Alternatively, the inhibitor may be a compound capable of suppressing the expression of EFHD2. According to embodiments of the present disclosure, 2-aryl propionic acid compounds (2-APAs) are preferred EFHD2 inhibitors. For cancerous cells having relatively higher level of endogenous EFHD2, reducing the expressed level of EFHD2 will slow down the growth of the cancer cells. Examples of the 2-APA compounds suitable for use in the present disclosure include, but are not limited to, ibuprofen, naproxen, flurbiprofen, and ketoprofen. In one preferred embodiment, the EFHD2 inhibitor is ibuprofen, which effectively suppresses the expression of EFHD2 in human non-small cell lung (NSCL) cancer cells, while non-steroid anti-inflammatory drugs (NSAID) such as aspirin, diclofenac, ketorolac, mefenamic acid, piroxicam, and sulindac, exhibit no such effect.

According to other embodiments, cancer cells pre-treated with the EFHD2 inhibitor (e.g., 2-APAs) are more susceptible to the treatment of an anti-cancer drug. In one preferred embodiment, human NSCL cancer cells pre-treated with ibuprofen are more susceptible to the action of the subsequently applied anti-cancer drug (e.g., cisplatin). Examples of the anti-cancer drug suitable for use in the present disclosure include, but are not limited to, cisplatin, carboplatin, oxalipatin, vinblastine, cyclophosphamide, methotrexate, 5-fluorouracil, vinorelbine, doxorubicin, docetaxel, bleomycin, dacarbazine, mustine, vincristine, procarbazin, prednisolone, epirubicin, and capecitabine. In another preferred embodiment, ibuprofen alone (25 mg/Kg, oral administration, three times per week) is more effective than cisplatin (5 mg/Kg, injection, once per week) in reducing the size of a tumor; and the combinational use of ibuprofen and cisplatin are even more effective in reducing the size of the tumor than either cisplatin or ibuprofen alone.

According to some embodiments of the present disclosure, overexpression of the EFHD2 polypeptide is found to correlate with the suppression of the expression of caveolin-1 (CAV1), suggesting that EFHD2 exerts its biological function through inhibition of CAV1. Thus, additionally or optionally, the method may further comprise the step of, administering to the subject another agent capable of activating the expression of caveolin-1 (CAV1). For example, a vector carrying CAV1 gene may be constructed and used as an agent to counteract the action of endogenous EFHD2 in a cancer patient, thereby preventing cancer cells of the primary tumor from metastasize to other locations in the body of the patient.

According to embodiments of the present disclosure, the subject is or has been afflicted with cancer, which may be any of breast cancer, gastric cancer, gastrointestinal stromal tumor (GIST), lung cancer (e.g., non-small cell lung cancer (NSCLC)), or pancreatic cancer. According to some embodiments of the present disclosure, the subject has gone through prior surgery to remove the cancer, such as NSCLC.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent.

### EXAMPLES

Materials and Methods

Cell lines and cell culture. Human lung adenocarcinoma cells A549 and H1299 were obtained from the American Type Culture Collection (ATCC, Maryland, USA). Human lung adenocarcinoma cell lines CL1-0 were from National Taiwan University Hospital, Taipei, Taiwan. Cell line CL1-5-F4 (F4) was established by selection of increasingly invasive cell populations from CL1-0 in according to procedures described by Chu Y W et al (Am J Respir Cell Mol Biol (1997) 17:353-360). H1299 and F4 cells were cultured in DMEM/F-12 media (Invitrogen). A549 and CL1-0 cells were maintained in RPMI 1640 media (Invitrogen). All culture media were supplemented with 10% fetal bovine serum and 1% antibiotics (GIBCO). All cells were grown in a humidified atmosphere of 5% CO₂ and 95% air at 37° C.

Animals. All procedures (2017-305) involving animal research were approved by the Institutional Animal Care and Use Committee (IACUC) at Chinese Medical University Hospital (CMUH) (Taichung, Taiwan). 5-week-old male BALB/c nude mice (18 to 25 g) were obtained from the National Laboratory Animal Center (Taipei, Taiwan). Mice were maintained in an environment under a 12: 12 hr light/dark cycle with access to food and water ad libitum.

Western blot analysis. Proteins were separated using 10% or 12.5% SDS-PAGE and transferred onto a PVDF membrane via electroblotting at 400V at 4° C. for 3 hr in 25 mmol/L Tris-HCl, 197 mmol/L glycine, and 13.3% (v/v) methanol. Then, 5% (w/v) skim milk in TBST was used for the blocking reaction, and the primary antibodies were incubated at room temperature overnight. After TBST washes, horseradish peroxidase-conjugated secondary antibodies were further incubated at room temperature for 1 hr. Immunoreactive signals were revealed using an enhanced ECL substrate according to the manufacturer's instructions (NEN Life Science). The primary antibodies used in this study included EFHD2 (ab106667; abcam), E-cadherin (#5296; Cell Signaling), vimentin (#3932; Cell Signaling), CAV1 (#3238; Cell Signaling), and β-actin (ab8226; abeam).

Immunohistochemical assay. Immunohistochemical assays (IHCs) were performed using an automatic BenchMark XT staining machine (Ventana Medical Systems) iVIEW 3,3-diaminobenzidine (DAB) detection kit (Ventana Medical Systems). Paraffin sections (4 μm) containing human lung adenocarcinoma tissues were deparaffinized, hydrated, and heated to 95-100° C. for 4 min to induce antigen retrieval. After inactivating endogenous peroxidase activity, rabbit anti-human EFHD2 polyclonal antibody (#ab119119; abeam; 1:1,200) was used to perform IHC staining. Tissue sections were finally incubated with iVIEW copper for 4 min to enhance signal intensity. Then, samples were counterstained with hematoxylin, dehydrated, mounted, and observed using an Eclipse E600 light microscope (Nikon). All staining results were evaluated by an experienced histologist.

Wound healing assay. The in vitro migration assay was performed using a Culture-Insert well (ibidi GmbH, German/v). Cancer cells (4.5×10⁴ cells) were cultured in suitable media in the device for 24 hr. After removal of the Culture-Insert, cancer cells were cultured for an additional 8 hr. The migration distance of cancer cells was recorded, and the migration area was measured using ImageJ software.

Matrigel invasion assay. For in vitro invasion assay, cancer cells (1.5×10⁵ cells in 200 μL) were suspended in the upper half of a PET membrane transwell insert chamber (BD Biosciences), which was coated with Matrigel (1 mg/mL; BD Biosciences), on a 24-well plate. Medium supplemented with 10% FBS was added as a chemoattractant to the lower half After incubation at 37° C. for 24 hr, cancer cells that passed through the insert were fixed with 3.7% formalin (Sigma-Aldrich) and stained with 0.1% crystal violet (Sigma-Aldrich).

MTT assay. MTT assay is a colorimetric assay that measures the activity of enzymes (i.e., reductase) that reduce (3-(4,5-dimethylthiazol-2yl)-2,5-diphenyltetrazoliumbromide (MTT), a yellow tetrazole, to purple formazan, in living cells. This reduction only takes place when cells are alive; hence MTT assay is generally used to assess the viability and proliferation of cells. Briefly, cells were challenged with various doses of the tested compound (e.g., ibuprofen and/or cisplatin at designated dose) for 24 hours. Then, MTT dye (500 μg/ml) was added and the reaction was allowed to proceed for 4 hours before being terminated by the addition of 500 μl of isopropanol. The absorbance of the solution at 570 nm was measured by spectrophotometer.

Construction of Short Hairpin Interference RNAs (shRNAs)

Nucleic acids that transcribed into small hairpin interference RNAs directed to the cleavage of the genes encoding EFHD2, GFP and CAV1, as listed in Table 2, were respectively constructed and synthesized. Negative control shRNA and target shRNA were provided from the National RNAi Core Facility (Academia Sinica, Taipei, Taiwan). The transcribed shRNAs were provided in Table 3.

TABLE 2
Nucleic Acids Transcribing the Present shRNAs

| | SEQUENCE (5′-3′) | SEQ ID |
|---|---|---|
| Name | Sense Strand | NO. |
| shEFHD2 | CCGGG AGAAG ATGTT CAAGC AGTAT CTCGA GATAC | 1 |
| | TGCTT GAACA TCTTC TCTTT TTTG | |
| shGFP | CCGGC AACAG CCACA ACGTC TATAT CTCGA GATAT | 2 |
| | AGACG TTGTG GCTGT TGTTT TTG | |
| shCAV1 | CCGGG ACGTG GTCAA GATTG ACTTT CTCGA GAAAG | 3 |
| | TCAAT CTTGA CCACG TCTTT TT | |

TABLE 3
Ribonucleic Acid Sequences of the present shRNAs

| | | SEQ ID |
|---|---|---|
| Name | SEQUENCE (5′-3′) | NO. |
| shEFHD2 | GGCCC UCUUC UACAA GUUCG UCAUA GAGCU CUAUG | 4 |
| | ACGAA CUUGU AGAAG AGAAA AAAC | |
| shGFP | GGCCG UUGUC GGUGU UGCAG AUAUA GAGCU CUAUA | 5 |
| | UCUGC AACAC CGACA ACAAA AAC | |
| shCAV1 | GGCCC UGCAC CAGUU CUAAC UGAAA GAGCU CUUUC | 6 |
| | AGUUA GAACU GGUGC AGAAA AA | |

RNA isolation and Quantitative polymerase chain reaction (qPCR). Total RNA was extracted with TRIzol reagent (Invitrogen). RT-PCR was performed using 1 μg of sample and the MMLV First-Strand synthesis kit (GeneDireX), and a ten-fold dilution of the RT-PCR product was applied for qPCR analysis. qPCR was performed using KAPA SYBR® FAST qPCR Master Mix Kit (Kapa Biosystems) by the LightCycler 480 apparatus (Roche). GAPDH served as an endogenous control. Specific DNA expression was estimated by the comparative Ct method using 2^{-ΔΔCT}.

Transfecting A549 Cells with the Synthetic shRNA Constructs

A549 cell line were maintained and cultured on 96-well plates, and were transfected with the plasmids carrying desired shRNAs the following day with the aid of the lipofectamine 2000 (Invitrogen, Carisbad, Calif. USA). Specifically, 5 μg peIF4G plasmid contained the present shRNAs (shEFHD2, shGFP or shCAV1) and 9 μl lipofectamine 2000 were allowed to form complexes in a period of 25 min at room temperature in antibiotic-free DMEM medium. The complexes were then added to A549 cells maintained and cultured in 96-well dishes and further incubated for another 24 hrs. For the selection of cells with high expression rate of shRNA, stable clones of human or murine shRNA expressed cells and negative control siRNA were selected by use of puromycin contained medium, and the selected clones were maintained in puromycin contained medium. The transfection of shRNAs in A549 cells was confirmed by the detection of target gene expression either in RNA level by RT-PCR analysis or in protein level by western blot assay.

Generation of Xenograft Mouse Model

BALB/c nude mice were orthotropically xenografted with H1299 cells (1×10⁶ cells) by stereotactic injection in the back. Mouse tumor progression (including growth and metastasis) was measured by IVIS weekly until week 7.

Statistical analysis. The data are displayed as the means±SD and categorical data are presented as frequencies and proportions. The significance of differences was examined by Student's t-test for continuous variables as well as Chi-square test or Fisher's exact test for categorical variables as appropriate. Overall survival and disease-free survival were determined by the Kaplan-Meier method and compared with using the log-rank test. Multivariable analysis of the independent factors associated with disease-free survival was performed using the Cox proportional hazard model. The statistical analysis of the data was performed using IBM SPSS Statistics 22 (IBM Co.). P<0.05 was considered statistically significant.

### Example 1 EFHD2 Promotes the Metastatic Abilities and Epithelial-to-Mesenchymal Transition (EMT) of Lung Adenocarcinoma Cells

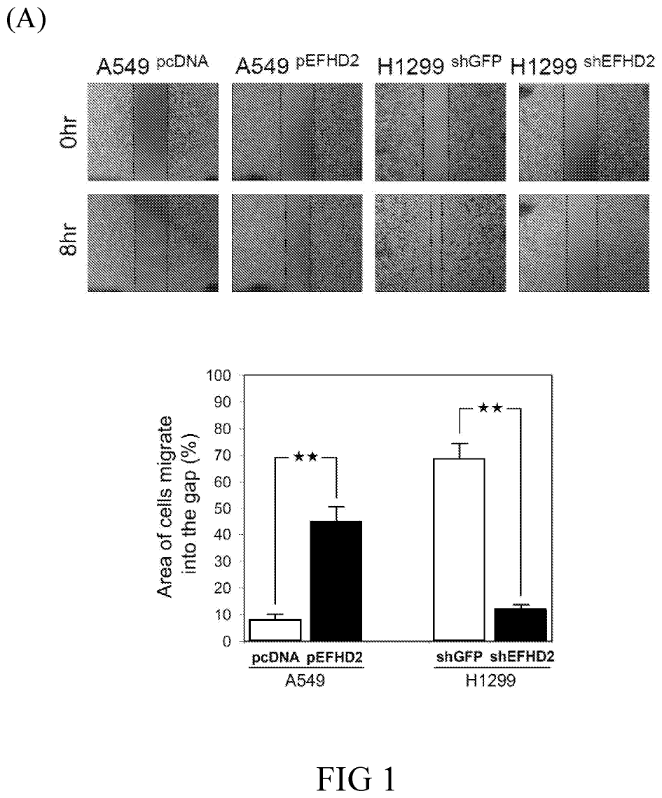

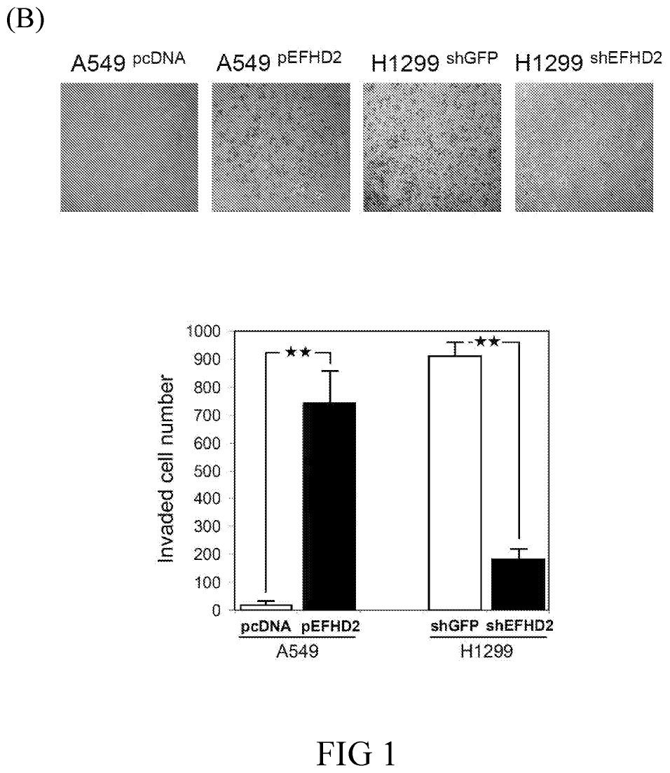

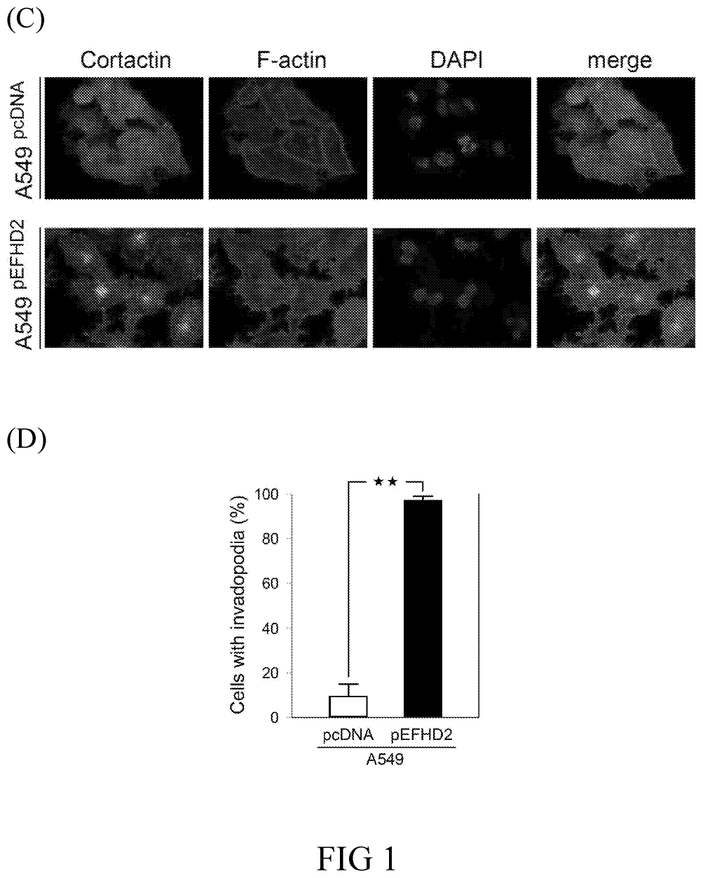

To determine whether EFHD2 contributes to the metastatic abilities of lung adenocarcinoma cells, the pcDNA vector was used to overexpress EFHD2 in A549 cells and shRNA (i.e., shEFHD2) to knockdown EFHD2 in H1299 cells. EFHD2 overexpression and knockdown did not obviously affect cell growth based on MTT assays. However, EFHD2 overexpression significantly increased migration and invasiveness in A549, whereas EFHD2 knockdown had the opposite effects in H1299, quantified results are respectively depicted in FIG. 1, panels (A) and (B).

A previous study indicated that EFHD2 modulates actin bundling and F-actin structure and contributed to the formation of lamellipodia (Huh Y. H. et al., Cell Mol. Life Sci. 70, 4841-4854 (2013)), which represents an important driver during metastasis. Accordingly, the effect of EFHD2 on lamellipodia formation was investigated using confocal microscopy. The images demonstrated that EFHD2 increased invadopodia-like protrusive structures and the formation of invadopodia, which can be visualized by colocalization of cortactin and F-actin (FIG. 1, panel (C)). In addition, EFHD2-overexpressing A549 cells exhibited significantly more cells with invadopodia structure compared with control cells (FIG. 1, panel (D)).

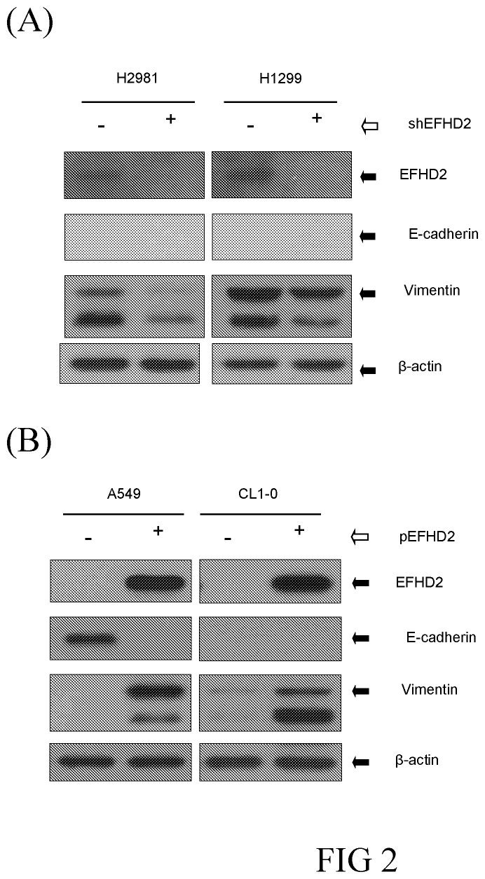

When tumor cells disseminate from the primary lesion to colonize distant organs, epithelial-to-mesenchymal transition (EMT) is an important step during the initiation of metastasis. To determine whether EFHD2 regulates the EMT program, the expression of EMT-related proteins after EFHD2 overexpression or knockdown was measured. Western blot assay indicated that EFHD2 knockdown decreased the expression of the mesenchymal cell marker vimentin in H1299 and H2981 cells (FIG. 2, panel (A)). By contrast, EFHD2 overexpression increased the expression of the mesenchymal cell marker vimentin and reduced the expression of the epithelial cell marker E-cadherin in A549 and CL1-0 cells (E-cadherin is undetectable in CL1-0) (FIG. 2, panel (B)). In addition, EFHD2 increased the expression levels of EMT-related transcriptional factors Snail, Twist1, ZEB1 and ZEB2 in A549 cells, but EFHD2 knockdown decreased the expression of these factors (data not shown).

Collectively, these results suggest that EFHD2 contributes to the promotion of EMT and metastasis in lung adenocarcinoma cells.

### Example 2 EFHD2 Promotes the EMT Through Inhibition of CAV1

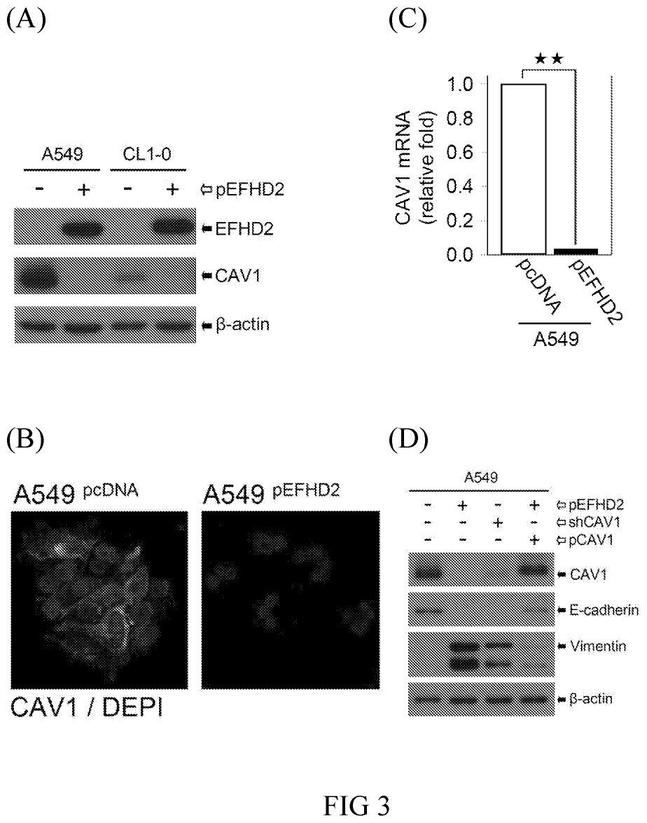

To investigate the underlying mechanism of EFHD2 in regulating EMT, a comparative proteomic analysis was performed to determine the protein signatures affected by EFHD2 overexpression. Total of 3,904 proteins were identified in EFHD2-overexpressing and control A549, and protein quantification was performed using label-free proteomic methods. Among these, 338 proteins exhibited more than 5-fold overexpression, and 383 proteins exhibited less than 0.2-fold expression in EFHD2-overexpressing A549 cells compared with control cells (data not shown). Given that EFHD2 is relevant to metastasis, a strategic combination search involving “the identified protein” and “metastasis” from the PubMed website was performed to identify the potential targets of EFHD2, which were validated by Western blot assay. It was found that the expression of caveolin-1 (CAV1) was significantly decreased if EFHD2 was overexpressed. EFHD2 dramatically suppressed CAV1 expression not only at protein levels, which were verified by Western blot and confocal microscopy assays (FIG. 3, panels (A) and (B)), but also at mRNA levels, which was determined by qPCR analysis (FIG. 3, panel (C)).

To confirm whether EFHD2 regulates the EMT through inhibition of CAV1, CAV1 knockdown in parental A549 and CAV1 rescue in EFHD2-overexpressing A549 cells were also performed. Direct CAV1 knockdown increased the expression of the mesenchymal cell marker vimentin, and decreases the expression of the epithelial cell marker E-cadherin (FIG. 3, panel (D)). In rescue experiments, the re-expression of CAV1 partly abolished EFHD2-induced EMT in A549 cells (FIG. 3, panel (D)). In addition, CAV1 knockdown enhanced the expression of EMT-related transcriptional factors Twist1, ZEB1 and ZEB2, which is similar to the effect of EFHD2 overexpression in A549 cells (data not shown).

Taken together, these results suggested that EFHD2 promotes the EMT through inhibition of CAV1.

### Example 3 2-APAs Suppress EFHD2 Expression in Cancer Cells

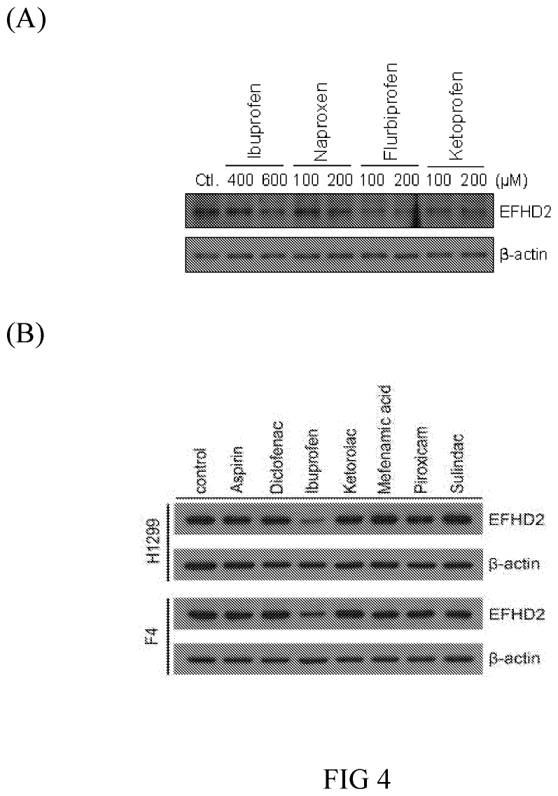

In this example, the respective effects of 2-APA compounds and non-steroid anti-inflammatory drugs (NSAID) on EFHD2 expression in H1299 and F4 cells were investigated. Results are illustrated in FIG. 4.

It was found that 2-APA compounds, including ibuprofen, naproxen, flurbiprofen, and ketoprofen were all effective in suppressing the expression of EFHD2 (FIG. 4, panel (A)), while the NASIDs, which include aspirin, diclofenac, ketorolac, mefenamic acid, piroxicam, and sulindac possessed no such effect (FIG. 4, panel (B)).

### Example 4 Effect of Ibuprofen on EFHD2 Expression in Cancer Cells

Based on the finding in Example 3, ibuprofen was chosen as an exemplified compound of 2-APA for further investigation on its effects on EFHD2 expression in cancer cells.

4.1 Ibuprofen Suppressed EFHD2 Expression and Cancer Cell Migration

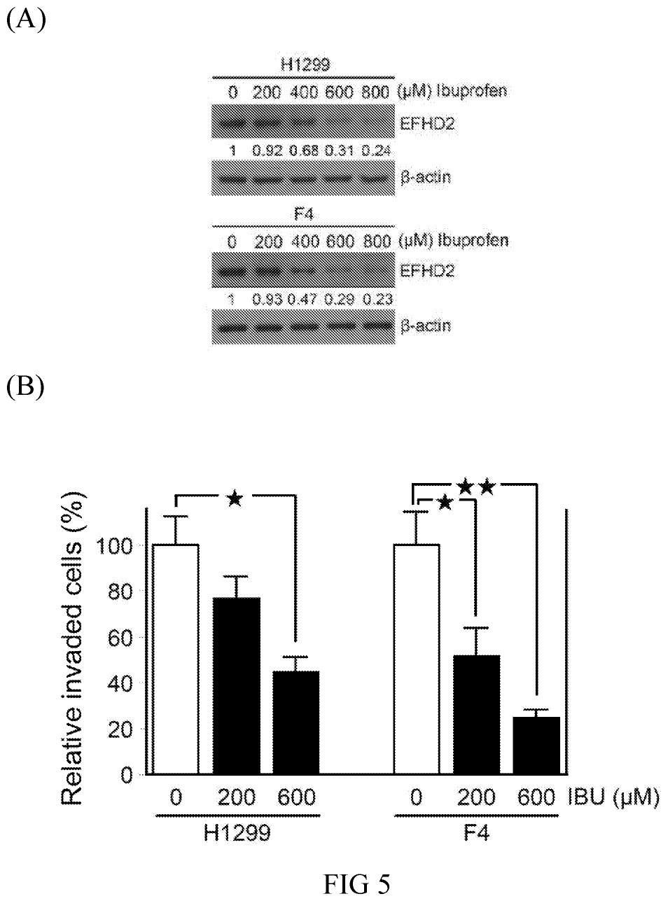

Reference is made to FIG. 5, in which ibuprofen was found to reduce the level of EFHD2 in a dose-dependent manner in both H1299 and F4 cells (FIG. 5, panel (A)); further, the migration and invasion abilities of H1299 and F4 cancer cells were also attenuated (FIG. 5, panel (B)).

4.2 Ibuprofen Activates Both Proteasomal and Lysosomal EFHD2 Degradation

To understand how ibuprofen suppresses EFHD2 expression, EFHD2 mRNA levels with or without ibuprofen treatment was first determined by qPCR. However, it was found that ibuprofen did not influence EFHD2 mRNA levels (data not shown).

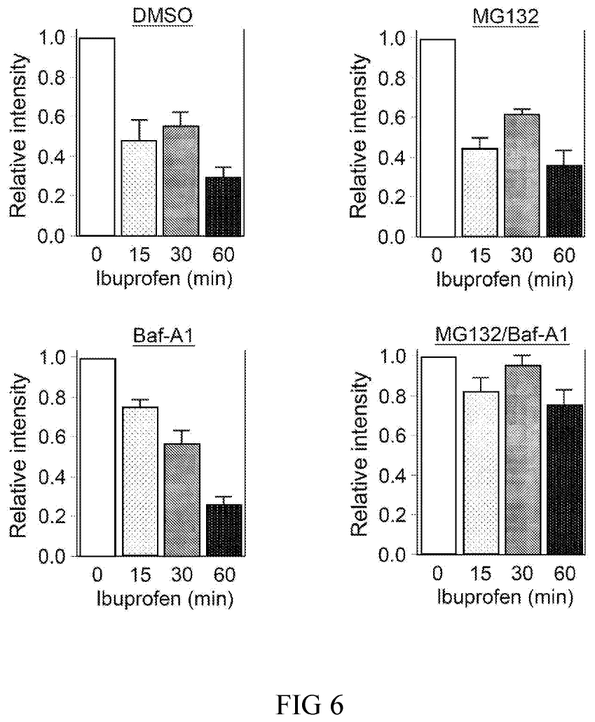

As ubiquitin-proteasome pathway and autophagy-lysosome pathway are two major systems responsible for cellular protein degradation, we then investigated whether any of the two systems has involved in ibuprofen-mediated EFHD2 degradation. To this purpose, proteasomal and autophagic protein degradation were respectively suppressed via use of MG132 and bafilomycin A1 (Baf-A1). It was found that MG132 or Baf-A1 alone was incapable of reducing ibuprofen-induced EFHD2 degradation, but combination of MG132 and Baf-A1 could stabilize EFHD2 (FIG. 6). The results strongly suggest that ibuprofen-induced EFHD2 degradation through both proteasomal- and lysosomal-dependent mechanism.

### Example 5 Ibuprofen Enhances Susceptibility of Cancer Cells to Chemotherapeutic Drug

5.1 Ibuprofen Enhances Susceptibility of Cancer Cells to Chemotherapeutic Drug Through Suppression of EFHD2

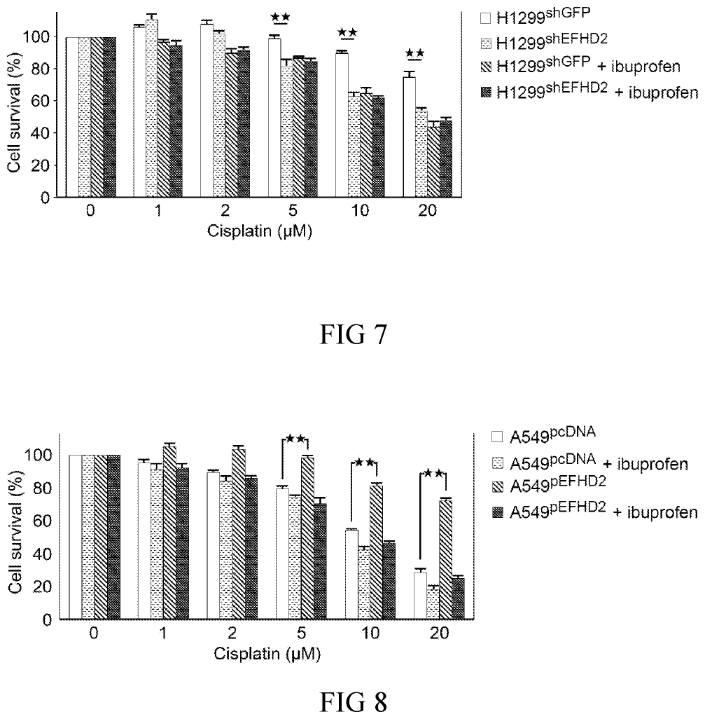

In this example, the mechanism of ibuprofen and its combination with a chemotherapeutic drug (i.e., cisplatin) were investigated via comparing their actions on the survival of H1299 cells in the presence or absence of EFHD2. To this purpose, H1299 cells were pre-treated with the present shRNA (i.e., H1299^{shEFHD2}) to knockdown the expression of EFHD2 in H1299 cells in accordance with procedures described in Example 1; and the control cells were pre-treated with shGFP. Results are depicted in FIG. 7.

Reference is first made to FIG. 7, as expected, the survival ratios of the normal H1299 cells (i.e., H1299^{shGFP}) and the EFHD2 knock-down H1299 cells (H1299^{shEFHD2}) both decreased with an increase in the concentration of cisplatin. Further, survival ratio of normal H1299 cells treated with the combination of ibuprofen (600 and cisplatin (1, 2, 5, 10 or 20 μM) (i.e., H1299^{shGFP}+ibuprofen) was lower than those treated with cisplatin alone (i.e., H1299^{shGFP}), which suggested that ibuprofen may enhance the susceptibility of H1299 cells to the subsequently applied cisplatin, resulting in lower survival ratio of the normal H1299 cells. However, for cells lacking EFHD2 expression, their survival ratios in the combined treatment of ibuprofen and cisplatin (i.e., H1299^{shEFHD2}+ibuprofen) were comparable to the control cells subjecting to the same treatment (i.e., H1299^{shGFP}+ibuprofen), which indicated that ibuprofen exerted its action through the inhibition of EFHD2.

5.2 Susceptibility of Cancer Cells Lacking Endogenous EFHD2 to Chemotherapeutic Drug is not Enhanced by Ibuprofen

In this example, instead of knocking down the endogenous EFHD2 expression via interference RNA, human lung adenocarcinoma cells A549 having low level of endogenous EFHD2 were used to further elucidate the action of ibuprofen (i.e., enhancing the susceptibility of cancer cells to chemotherapeutic drugs). Results are illustrated in FIG. 8.

As expected, lacking endogenous EFHD2 led to no enhanced susceptibility by ibuprofen to cisplatin treatment (A549^{pcDNA} vs A549^{pcDNA}+ibuprofen), and supplementing EFHD2 via transfecting A549 cells with vectors carrying exogenous EFHD2 genes resulted in A549 cells being more resistant to cisplatin treatment (A549^{pcDNA} vs A549^{pEFHD2}). However, treating A549 cells having exogenous EFHD2 with ibuprofen failed to increase their susceptibilities to cisplatin (A549^{pEFHD2} vs A549^{pEFHD2}+ibuprofen), which might be due to the overexpressed level of EFHD2 in A549 cells, resulting in failing to suppress EFHD2 via ibuprofen treatment.

### Example 6 2-APAs Sensitize Cancer Cells to Chemotherapeutic Drug

In this example, the effect of 2-APAs (e.g., ibuprofen, flurbiprofen, naproxen and ketoprofen) and cisplatin on F4 cells were investigated.

Similar to the findings in Example 5, when lung cancer F4 cells were pre-treated with 2-APAs (20 μM), including buprofen, flurbiprofen, naproxen and ketoprofen, they became more sensitive to the subsequent treatment of cisplatin, resulting more cell death as compared to the control (data not shown).

### Example 7 Synergistic Reduction in Tumor Size Via the Combined Treatment of Ibuprofen and Cisplatin

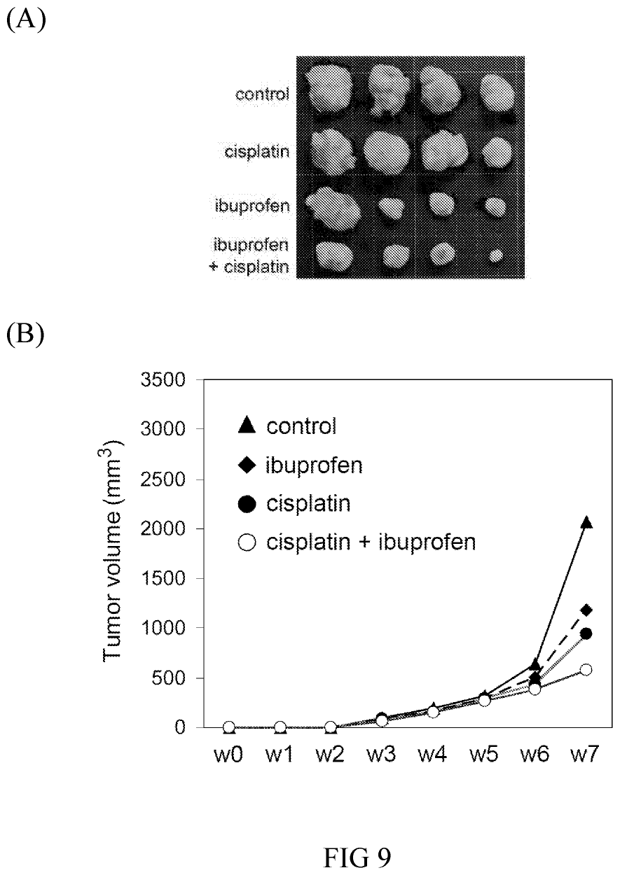

In this example, in vivo effect of the combined treatment of ibuprofen and cisplatin was investigated via the xenograft mouse model established in accordance with the procedures described in the “Materials and methods” section. For treatment purpose, mice were treated with cisplatin (5 mg/Kg, via injection, once per week for 3 weeks) and ibuprofen (25 mg/Kg, orally fed, on the day when cisplatin was injected, and 1 day prior to, and 1 day after the cisplatin injection), alone or in combination. Mice were then sacrificed and the tumors were isolated and further analyzed. Results are depicted in FIG. 9.

As expected, cisplatin treatment could significantly reduce the size of the tumor, ibuprofen alone also possessed such effect, but was more effective than cisplatin. Surprisingly, the combined use of cisplatin and ibuprofen resulted in synergistically reduction in the tumor size.

Taken together, the present disclosure identifies and confirms that EFHD2 is closely associated with the growth and metastasis of a tumor. Accordingly, an agent (either an interference RNA or a compound) capable of suppressing the expression of EFHD2 could serve as a lead compound for the development of a medicament for preventing tumor cells from growing and/or metastasizing.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A method of treating a subject afflicted with a metastatic cancer mediated by the expression of EF-hand domain-containing protein D2 (EFHD2) comprising administering to the subject an effective amount of 2-aryl propionic acid (2-APA) or a pharmaceutically acceptable salt thereof.

2. The method of claim 1, wherein the 2-APA is selected from the group consisting of ibuprofen, naproxen, flurbiprofen, and ketoprofen.

3. The method of claim 2, wherein the 2-APA is ibuprofen.

4. The method of claim 1, wherein the metastatic cancer is any of breast cancer, gastric cancer, gastrointestinal stromal tumor (GIST), lung cancer, or pancreatic cancer.

5. The method of claim 4, wherein the lung cancer is non-small cell lung cancer.

6. An isolated double stranded short hairpin ribonucleic acid (shRNA) that directs cleavage of an EF-hand domain-containing protein D2 (EFHD2) gene RNA via RNA interference, wherein one strand of said shRNA molecule comprises a ribonucleic acid complementary to said EFHD2 gene RNA encoded by a nucleic acid of SEQ ID NO: 1 or a portion thereof.

7. The isolated double stranded shRNA of claim 6, wherein the shRNA has the ribonucleic acid at least 90% identical to SEQ ID NO: 4.

8. A method for treating a subject afflicted with a metastatic cancer comprising administering to the subject an effective amount of an agent capable of suppressing the expression of EFHD2 gene in the metastatic cancer of the subject.

9. The method of claim 8, wherein the agent is a shRNA that directs the cleavage of a ribonucleic acid of said EFHD2.

10. The method of claim 9, wherein the shRNA comprises a ribonucleic acid complementary to said ribonucleic acid of EFHD2 gene encoded by a nucleic acid of SEQ ID NO: 1 or a portion thereof.

11. The method of claim 10, wherein the ribonucleic acid of the shRNA is at least 90% identical to SEQ ID NO: 4.

12. The method of claim 8, wherein the agent is an inhibitor specific to said EFHD2.

13. The method of claim 12, wherein the inhibitor is an antibody or an aptamer capable of binding selectively to said EFHD2, and the aptamer is identified through systematic evolution of ligands by exponential enrichment (SELEX).

14. The method of claim 12, wherein the inhibitor is a 2-aryl propionic acid (2-APA) selected from the group consisting of ibuprofen, naproxen, flurbiprofen, and ketoprofen.

15. The method of claim 14, wherein the inhibitor is ibuprofen.

16. The method of claim 8, further comprising administering to the subject another agent capable of evoking the expression of caveolin-1 (CAV1).

17. The method of claim 8, wherein the cancer is any of breast cancer, gastric cancer, gastrointestinal stromal tumor (GIST), lung cancer, or pancreatic cancer.

18. The method of claim 17, wherein the lung cancer is non-small cell lung cancer (NSCLC).

19. The method of claim 8, wherein the subject has gone through prior cancer removal surgery.

20-24. (canceled)


SEQUENCE LISTING (6 sequences)

SEQ ID NO: 1 - DNA, 59 bp, Artificial Sequence
  shEFHD2; synthetic
ccgggagaagatgttcaagcagtatctcgagatactgcttgaacatcttctcttttttg

SEQ ID NO: 2 - DNA, 58 bp, Artificial Sequence
  shGFP; synthetic
ccggcaacagccacaacgtctatatctcgagatatagacgttgtggctgttgtttttg

SEQ ID NO: 3 - DNA, 57 bp, Artificial Sequence
  shCAV1; synthetic
ccgggacgtggtcaagattgactttctcgagaaagtcaatcttgaccacgtcttttt

SEQ ID NO: 4 - RNA, 59 nt, Artificial Sequence
  shEFHD2; synthetic
ggcccucuucuacaaguucgucauagagcucuaugacgaacuuguagaagagaaaaaac

SEQ ID NO: 5 - RNA, 58 nt, Artificial Sequence
  shGFP; synthetic
ggccguugucgguguugcagauauagagcucuauaucugcaacaccgacaacaaaaac

SEQ ID NO: 6 - RNA, 57 nt, Artificial Sequence
  shCAV1; synthetic
ggcccugcaccaguucuaacugaaagagcucuuucaguuagaacuggugcagaaaaa